(19) United States
(12) Plant Patent Application Publication
(10) Pub. No.: US 2003/0033648 P1
(43) Pub. Date: Feb. 13, 2003

(54) Hydrangea macrophylla plant named 'Frau Mariko'

(75) Inventor: Hiroshi Ebihara, Tochigi-ken (JP)

Correspondence Address: CHRISTIE, PARKER & HALE, LLP, P.O. BOX 7068, PASADENA, CA, 91109-7068, US

(73) Assignee: MIYOSHI & CO., LTD.

(21) Appl. No.: 10/267,721

(22) Filed: Oct. 8, 2002

Related U.S. Application Data
(63) Continuation of application No. 09/676,603, filed on Sep. 28, 2000, now abandoned.

(51) Int. Cl. A01H 5/00

(52) U.S. Cl. PLT/250

(57) ABSTRACT

A new plant variety of Hydrangea macrophylla by the combination of the attractive pigmentation pattern of its sepals which are predominately colored with narrow, irregular white edges; its deeply ruffled and serrated sepal edges and dense its inflorescence on a strong stem that does not require staking for support.

### CROSS-REFERENCE TO RELATED APPLICATION

The present application is a continuation application of U.S. patent application Ser. No. 09/676,603, filed Sep. 28, 2000, entitled “PLANT HYDRANGEA MACROPHYLLA NAMED ‘FRAU MARIKO’”, the disclosure of which is incorporated herein by reference.

### BACKGROUND OF THE INVENTION

The present invention relates to a new and distinct variety of Hydrangea macrophylla (Thunb.) Ser. Which was developed in a controlled breeding program in Ninomiya-machi Prefecture, Japan by Mr. Hiroshi Ebihara. The varietal denomination of the new variety is ‘Frau Mariko’.

The genus Hydrangea is included in the family Saxifragaceae which comprises about 80 genera and 1,200 species of herbs, shrubs, small trees, woody climbers mostly temperate in origin. Hydrangea comprises approximately 23 species of deciduous or evergreen shrubs and vines originating in North America, South America and East Asia, many of which possess desirable ornamental characteristics. Recent taxonomical treatments sometimes isolate Hydrangea in a new family, Hydrangeaaceae.

Hydrangea macrophylla is widespread and common throughout much of Japan. It generally has opposite, simple leaves, obovate to elliptic in shape, 10 to 20 cm long and 5 to 15 cm wide. Leaf color ranges from light to dark green, depending on light intensity and cultural conditions. Flower color is dependent upon the pH of the soil and the amount of available aluminum in the soil media. Leave are generally softly pubescent. Flowers of Hydrangea macrophylla are borne in mostly sterile florets, formed in what is often called a ‘hortensia’ flower.

### SUMMARY OF THE INVENTION

The new variety was discovered in a controlled breeding program of Hydrangea macrophylla and differs from its parents in the combination of the attractive pigmentation pattern of its sepals which are predominately colored with narrow, irregular white edges, its deeply ruffled and serrated sepal edges, and its dense inflorescence on a strong stem that does not require staking for support. Asexual reproduction of the new variety by stem cuttings performed in Ninomiya-machi Prefecture, Japan; Lewisberry, Pa. and Vacaville, Calif.; have confirmed that the distinctive characteristics of the new variety are stable and transmitted to succeeding generations, and the new variety reproduces true to type.

### COMPARISON WITH PARENT

‘Frau Mariko’ is distinguished from its parent and all other varieties of Hydrangea macrophylla of which I am aware by the combination of the attractive pigmentation pattern of its sepals which are predominantly colored with narrow, irregular white edges, its deeply ruffled and serrated sepal edges, and its dense inflorescence on a strong stem that does not require staking for support.

### BRIEF DESCRIPTION OF ILLUSTRATIONS

The accompanying photographic illustrations show a specimen of the new cultivar as true to color as is reasonably possible to make in an illustration of this character.

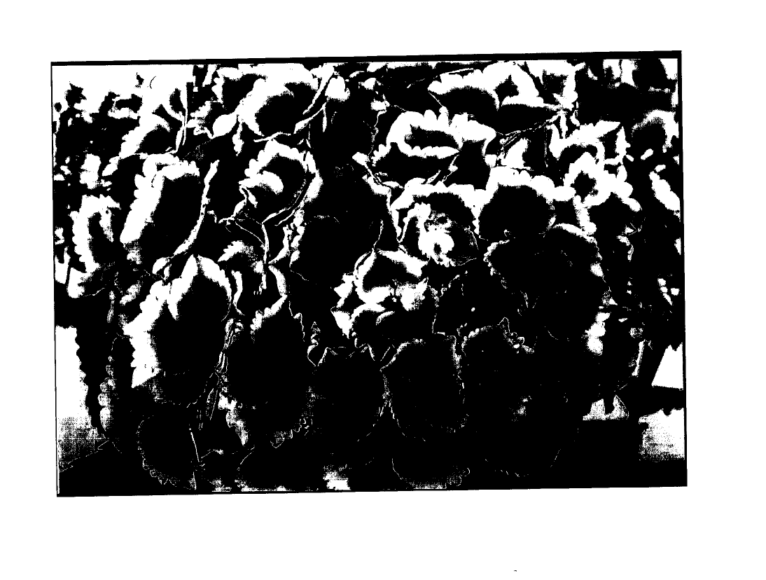

FIG. 1 illustrates the inflorescence with distinct serrated sepals; narrow white edge on the flower margin and unique flower color.

### DETAILED DESCRIPTION OF THE NEW VARIETY

‘Frau Mariko’ has not been observed under all possible environmental, cultural, and light conditions. The following observations and descriptions are of 2-year-old plants grown in 5-gallon containers in Lewisberry, Pa. In this description, color references are to The Royal Horticultural Society Colour Chart (1995) and terminology used in the color descriptions herein refers to plate numbers in this color chart. Phenotypic expression may vary with light intensity, cultural, and environmental conditions.

### CLASSIFICATION

Botanical: Hydrangea macrophylla (Thunb) Ser. ‘Frau Mariko’

Parentage:

Mother plant.—Hydrangea macrophylla ‘Crystal’(unpatented).

Father plant.—Hydrangea macrophylla ‘Silver Edge’ (unpatented).

Propagation: By vegatative cuttings

Time to initiate rooting: Approximately 2 weeks

### PLANT

Size: Mature plant can grow to approximately 1 m tall and 1 m wide

Branching: Freely on one-year-old wood

Habit: Upright, mound forming deciduous shrub

Stems:

Length.—To 1 m.

Diameter.—Approximately 1 cm at base of plant.

Color.—Near Yellow-Green Group 145C.

Leaf shape.—Elliptic with acute base and apex.

Leaf margin.—Serrate.

Leaf texture.—Thick; glabrous top and underside; veins prominent on the underside of the leaf and are furrowed on the leaf surface.

Internodes.—From 1 to 5 cms.

Leaf color.—Upper side: From near Green Group 137A to 137B Under side: Near Yellow-Green Group 145C.

Petioles.—Approximately 24 mm long.

Leaf size.—To about 105 mm long and 72 mm wide.

Texture and substance: Glabrous

Hardiness: USDA Zone 5 (−20° to −10° F.)

Pests/Diseases.—No pest or disease problems noted.

### INFLORESCENCE

Bloom period.—Spring to Summer.

Flower form.—Paniculate, both sterile, sepalous florets and fertile, asepalous florets borne on the same panicle.

Flower arrangement.—Rounded Flower size: Approximately 20 cm.

Quantity of florets per cluster.—Approximately 135 florets per cluster Color: Sepal color is near Blue Group 104A at margins; ranging from Blue Group 105A-D approaching the center of sepal; and near Blue Group 106C at center of sepal; sepal color can vary depending on soil pH and nutritional amendments supplied.

Shape of inflorescence.—Spherical clusters of numerous small florets are curved and the sepals of the floret overlap one another. Sepals elongate and become colored as the inflorescence matures.

Size of inflorescence.—The individual sterile florets are variable in size, but can measure up to 4 cm in diameter.

Pedicels:

Length.—2 to 2.5 cms.

Diameter.—Less than 1 mm.

Color.—Near Blue Group 104A.

Peduncles:

Length.—7 to 10 cms.

Diameter.—Approximately 0.5 cm.

Color.—Near Yellow-Green Group 145C.

Fragrance: None noted

### REPRODUCTIVE ORGANS

Stamens: 7 to 9 stamens. Pollen is white

Stigma: One three-pronged stigma

Fruit: Minute in appearance, less than 1 mm long and wide

Sepalous florets:

Number of sepals.—4; occasionally with a much reduced 5^{th} sepal.

Aspect of sepals.—Flower strongly cupped, with broadly overlapping sepals; sepals often strongly sulcate; occasionally twisted at the apex.

Size of sepals: Approximately 1 mm long and 1 mm wide

Shape of sepals: Ovate

Margins: Strongly serrated

I claim:

1. A new plant of Hydrangea macrophylla of the variety substantially as shown and described.
